(19) United States
(12) Patent Application Publication
(10) Pub. No.: US 2015/0351824 A1
(43) Pub. Date: Dec. 10, 2015

(54) TEST FIXTURE FOR AN ELECTROSURGICAL DEVICE

(71) Applicant: Medtronic, Inc., Minneapolis, MN (US)

(72) Inventors: Roger D. Greeley, Portsmouth, NH (US); Brian M. Conley, South Berwick, ME (US); Duane W. Marion, Portsmouth, NH (US)

(73) Assignee: Medtronic, Inc., Minneapolis, MN (US)

(21) Appl. No.: 14/297,102

(22) Filed: Jun. 5, 2014

(51) Int. Cl. A61B 18/12; G01L 5/00; A61B 19/00

(52) U.S. Cl. CPC A61B 18/12; A61B 19/46; G01L 5/0028; A61B 2018/00595

(57) ABSTRACT

The present disclosure provides a system including a test fixture for an electrosurgical device. Embodiments of the test fixture may include a tissue carriage for mounting or loading a tissue sample thereon, a device mount for mounting a test device, a linear actuator for regulating a tissue incision or therapy speed and a force transducer for measuring a tissue incision or therapy force.

### FIELD

The present disclosure relates generally to the field of medical devices including electrosurgical devices, systems and methods that provide for cutting, coagulation, hemostasis and sealing of tissue. More particularly, this disclosure relates to test fixtures for electrosurgical devices including test fixtures that regulate parameters of a test incision.

### BACKGROUND

One of the critical parameters of an electrosurgical incision is the resultant thermal spread into the tissue being cut. The speed of the incision can have a significant influence on the thermal spread. For example, a slower cut may result in a deeper thermal spread due to the increased length of time a device is placed on tissue. If the speed of the incision is regulated or known, comparisons of thermal spread may be made more accurately.

Likewise, if the location of the test or tissue sample relative to the location of the effective end of the device, for example an electrode or cutting end, is regulated or known, analysis of the effects of the device on the tissue may be more accurate or may provide better insight into the device functionality and capability. Along these lines, when testing the effects of a device on tissue, a tissue sample may be mounted in a test fixture. Pulling taut or applying tension to a tissue sample while cutting or otherwise testing the tissue may provide a more clinically accurate mode of cutting. Furthermore, measurements of the force required to cut tissue may beneficially show how easy or difficult a device cuts through tissue.

### BRIEF DESCRIPTION OF THE DRAWINGS

FIG. 1 is a perspective view of a system including a test device and a test fixture having a tissue carriage shown in a first position, according to an embodiment.

FIG. 2 is a perspective view of the test fixture of FIG. 1 showing the carriage in a second position, according to an embodiment.

FIG. 3 is a side view of the system of FIG. 1, with the controller and power not shown.

FIG. 4 is an exploded view of a tissue carriage having a tissue cartridge and a housing, according to an embodiment.

FIG. 5 is a perspective view of the tissue cartridge of FIG. 4 rotated 180 degrees.

FIG. 6 is a cross-sectional view of the tissue carriage of FIG. 3 in assembled form, according to an embodiment.

FIGS. 7A-D depict tissue loading steps according to a method of the disclosure.

### DETAILED DESCRIPTION

Throughout the description, like reference numerals and letters indicate corresponding structure throughout the several views. Also, any particular feature(s) of an exemplary embodiment may be equally applied to any other exemplary embodiment of this disclosure. In other words, features between the various exemplary embodiments described herein may be interchangeable as suitable, and not exclusive. In addition, reference to a tissue or tissue sample likewise is intended to refer to any test sample or material desired to be tested or placed in a test fixture of the disclosure. Furthermore, from the disclosure it should be clear that use of the terms “distal” and “proximal” are made in reference to the user of a device.

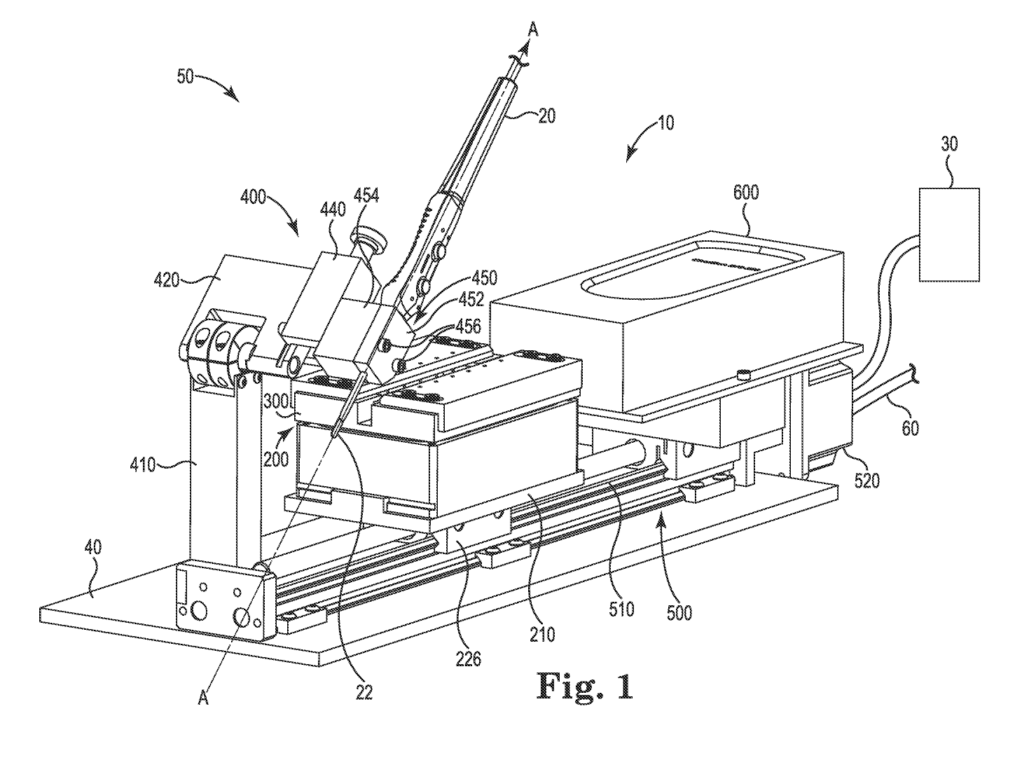

FIG. 1 is a perspective view of a system 50, according to an embodiment. System 50 represents one embodiment of a system according to the disclosure and includes a test fixture 10, a test device 20, a controller 30, power connector 60 and an optional test fixture base 40. The test fixture 10, according to the embodiment of FIG. 1, includes a tissue carriage 200, device mount 400, linear actuator 500, and a force transducer 600. In some embodiments, system 50 and/or test fixture 10 do not include a force transducer 600.

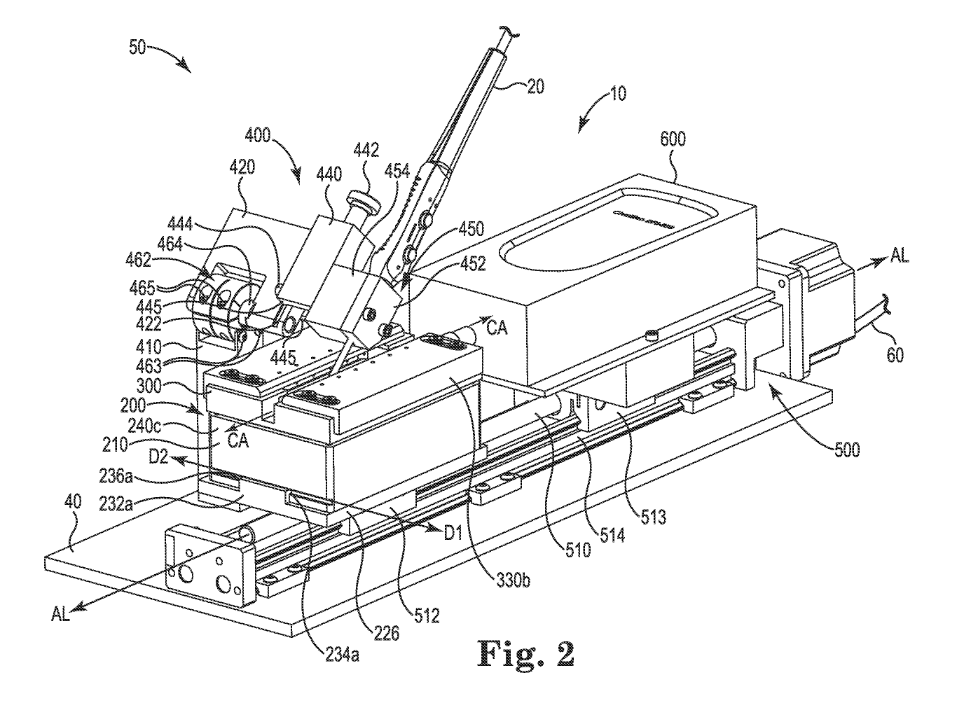

As shown in FIGS. 1-3, a test device 20 is mounted in the device mount 400. Device 20 can be any of a number of devices including various types of electrosurgical devices. For example, device 20 may be a device which provides for cutting, coagulation, hemostasis and/or sealing of tissue. Regardless of the particular device desired to be tested, the device mount 400 includes a device mounting block 450 which may be removably attached to a device height adjuster 440. Mounting block 450 can be sized to receive a device 20 therein. Various sizes of mounting blocks 450 can be made to accommodate various sized devices 20. Mounting of the device 20 can be achieved via various retention mechanisms as known in the art. For example, and as shown in the embodiment of FIGS. 1-2 mounting block 450 includes a first device holder plate 452 and a second device holder plate 454. An interior (not shown) of each plate 452, 454 may be sized to receive a particular device 20, and more specifically, a distal, electrode or procedurally effective end 22 of a device 20. Thus, the interior of plate 452 can include a depression or other feature sized to receive device 20 while an interior of plate 454 can include a similar or mating depression or feature sized to receive the device 20. Plates 452, 454 include plate attachment pins 456 which can take the form of a screw or other fasteners as known in the art. Pins 456 are configured to mate with holes in plate 454 such that tightening of the pins 456 adjustably engages the distal end 422 of device 20 between the plates 452, 454. In this manner, plates 452, 454 can surround a device 20 placed therein in and when matingly connected can fixedly hold a device 20 (i.e., device 20 can be held stationary and will not slip or move along an axis A (FIG. 1) parallel to the device 20).

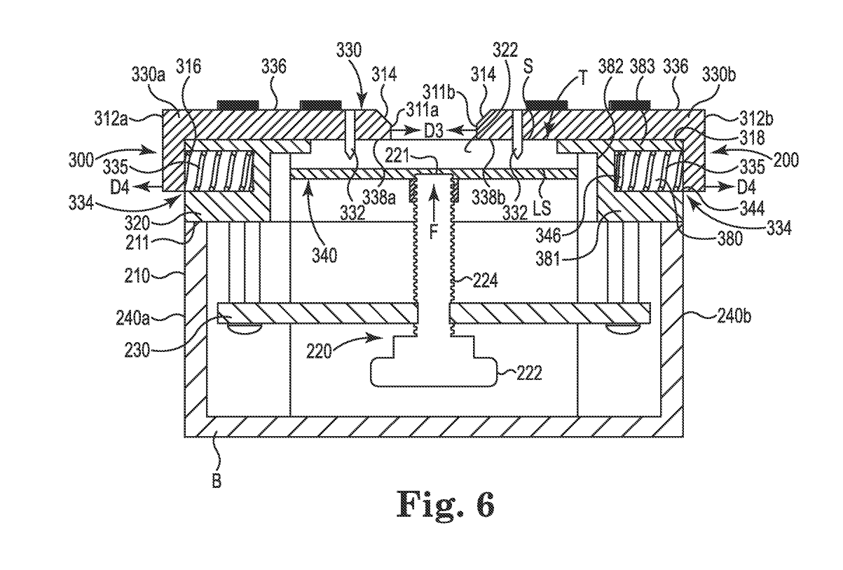

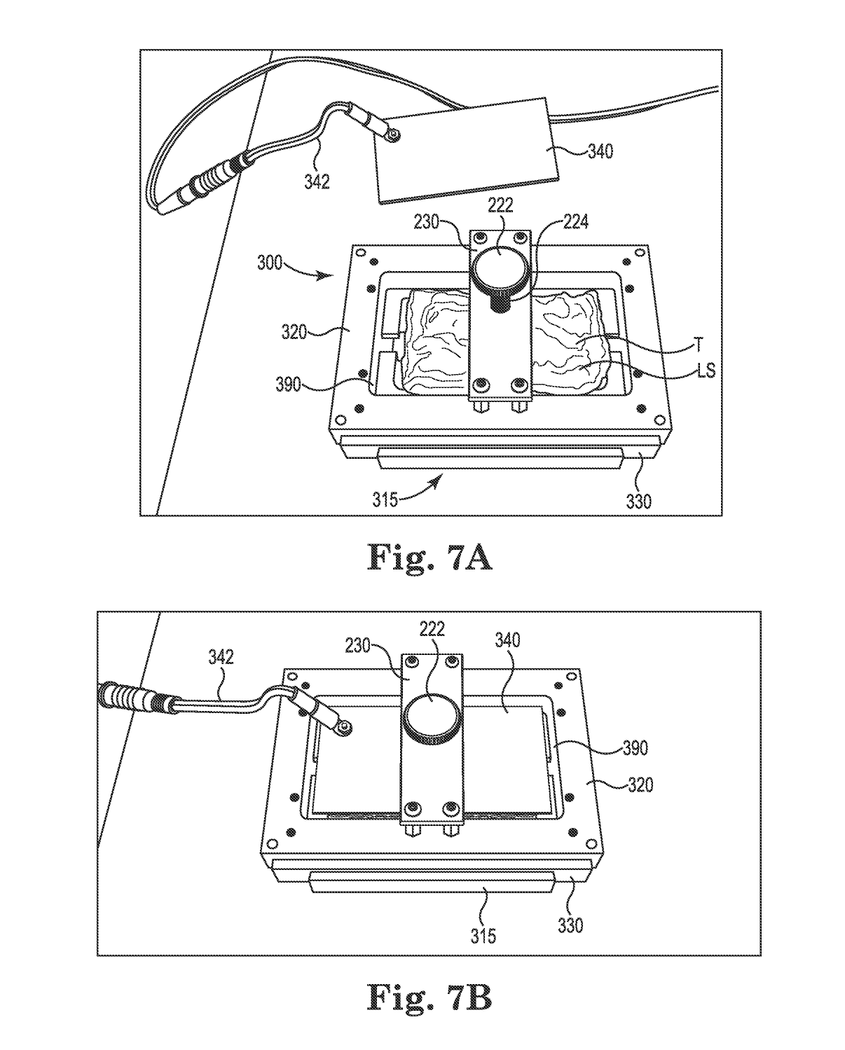

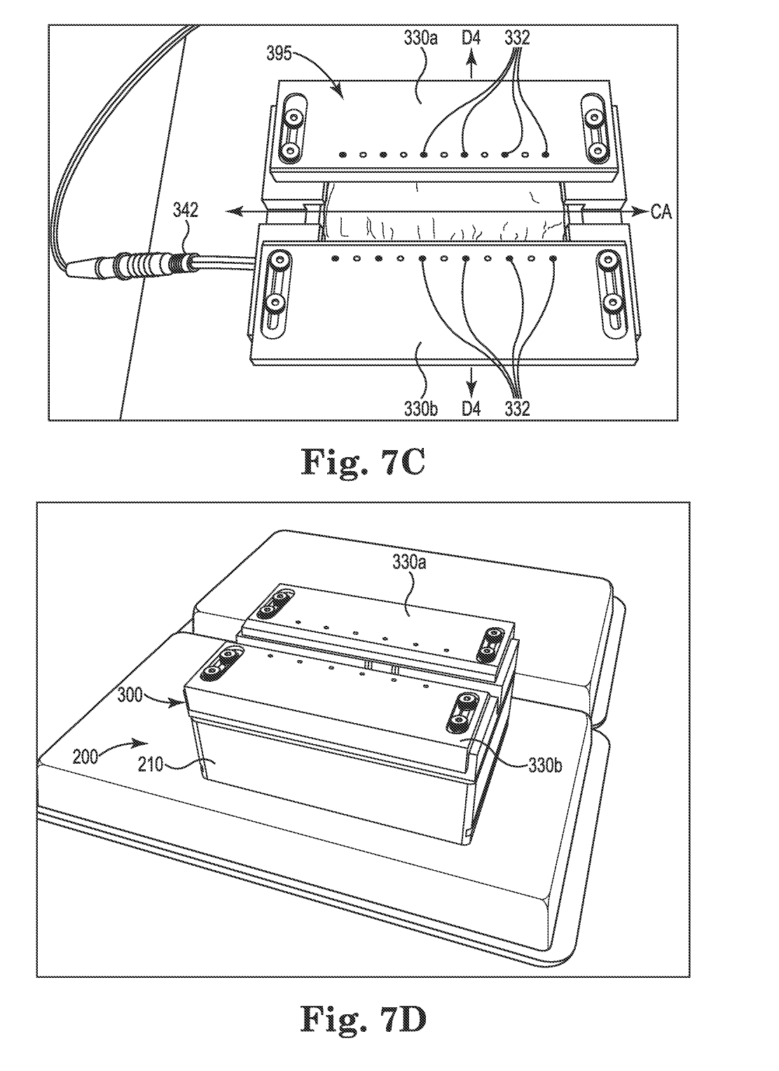

A device height adjuster 440 can adjust the height of the device 20 relative to the tissue carriage 200, when the device 20 is held by the device mounting block 450. The device mounting block 450 may be removably connected to the device height adjuster 440 via screw 444, or other attachment mechanism. The height adjuster 440 includes arms 445 configured to slide within an adjuster plate track 422 (FIG. 2) provided on a device adjuster plate 420. The track 422 allows movement of the device height adjuster 440 relative to the adjuster plate 420 and along axis A. A height adjuster knob 442 is connected to the device height adjuster 440 and is threadably coupled to the height adjuster plate 420. Accordingly, when the height adjuster knob 442 is turned (such as by the hand of a user) the arms 445 of the device height adjuster 440 move within track 422 causing the device height adjuster 440 to move relative to the adjuster plate 420. Movement of the device height adjuster 440 in turn causes movement of a device 20 along an axis parallel to axis A, when the device 20 is held in the device mounting block 450. As will be discussed further herein, this movement allows for adjustment of the effective end 22 of device 20 such that end 22 may be positioned at a desired location or position relative to a test sample or tissue T (FIGS. 6, 7A).

The device adjuster plate 420 is rotatably attached to an arm 410 via a split clamp 462. Split clamp 462 is coupled to the arm 410 via screws 463 and is rotatably coupled to a clamp pin 464. The clamp pin 464 is in turn coupled to the device adjuster plate 420. Attachment of the adjuster plate 420 in this manner allows for at least about 180 degrees of rotation of the plate 420 about a pin (not shown) of the split clamp 462, when the tissue carriage 200 is not mounted to the actuator 500. When the tissue carriage 200 is mounted to the actuator 500, rotation of the plate 420 about the clamp pin 464 is limited only by the carriage 200. Clamp 462 may be incorporated into arm 410, i.e., not removable or may be attached via the screws 463 or other attachment mechanisms as known in the art. The adjuster plate 420 can be rotated to a desired rotational position and fixed or held stationary via engagement of screws 465 with the clamp pin 464. Tightening of the screws 465 can effectively set the clamp 420 at the desired angle. As can be seen in FIGS. 1 and 2, rotation of device adjuster plate 420 causes the attached device mount 400 to rotate therewith and thus allows a device 20 to likewise rotate when the device 20 is held by device mount 400.

As described above, since mounting block 450 can fixedly hold a device 20, and the device adjuster plate 420 can fixedly hold the mounting block 450 at a desired angle, the end 22 of a device 20 can thus be maintained at a fixed angle and distance from (i.e., at a fixed position or location relative to) a test sample or tissue T.

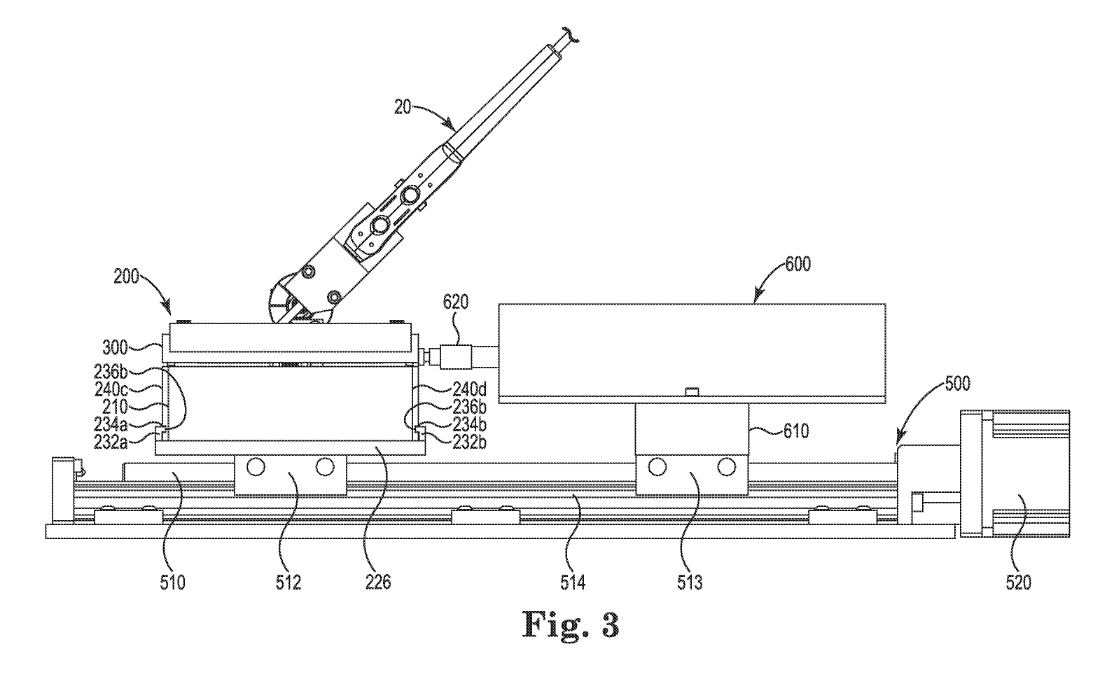

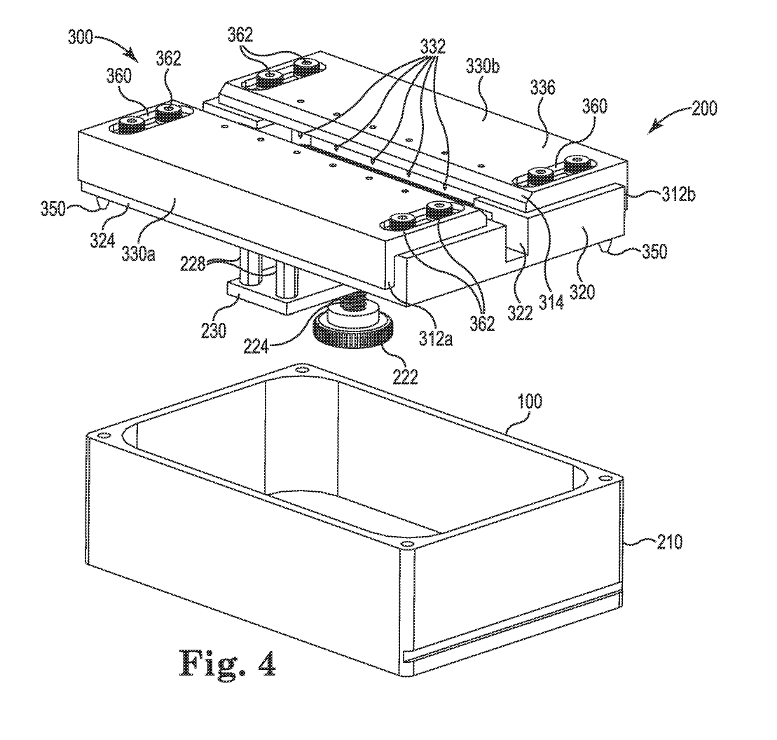

As depicted in FIGS. 1-3, test fixture 10 includes a tissue carriage 200. Tissue carriage 200 includes a tissue cartridge 300 and a housing 210 having a cavity 100. Tissue cartridge 300 is removably connected or separably attached to (i.e., detachable from) housing 210. In addition, the tissue carriage 200 may be removably connected or separably attached to a first actuator travel carriage 512 that is slidably connected to an actuator shaft 510. The tissue carriage 200 may be separably attached to the first actuator travel carriage 512 via a tissue carriage base 226 adapted to receive and hold the carriage 200. For example, and as best seen in FIGS. 2-3, tissue carriage base 226 is provided on the first actuator travel carriage 512 and includes two upstanding brackets, 232a, 232b (also, collectively “brackets 232”) which may be provided as L-shaped brackets as one example, including an arm or member 234a, 234b (also, collectively “arms 234”) which cooperate with a groove 236a, 236b (also, collectively “grooves 236”) provided on opposed third and fourth sides 240c, 240d (also, collectively with sides 240a, 240b, “sides 240”), of housing 210. Removal of the tissue carriage 200 from the base 226 may therefore be achieved by sliding the tissue carriage 200 in a direction D1 or D2 (either forward or rearward as shown) that is, in a direction perpendicular to an axis AL of the actuator shaft 510. Other removable or quick-connect type attachment mechanisms are contemplated and the “tongue in groove” attachment described above is merely illustrative of one embodiment of the present disclosure. Alternatively, the tissue carriage 200 and specifically the housing 210 may be fixedly attached to the first actuator travel carriage 512.

With reference between FIGS. 4 and 6, the tissue cartridge 300 is provided at an upper end 211 of the housing 210 of the tissue carriage 200. The tissue cartridge 300 is removably attached (i.e., separably connected) to the housing 210 via housing attachment elements 350 which may be screws, pins, clips or other attachment mechanisms. FIG. 4 shows the tissue cartridge 300 detached from the housing 210. When separated or detached from the housing 210, the tissue cartridge 300 allows for bottom loading of tissue a T, as will be explained in further detail below. The tissue cartridge 300 includes first and second doors 330a, 330b (also, collectively “doors 330”), a return plate adjuster 220, an adjuster positioning plate 230, one or more adjuster positioning plate anchors 228 (a plurality of anchors 228 being shown in FIGS. 4-6), one or more tissue engagement mechanisms 332 (a plurality of engagement mechanisms 332 being shown in FIGS. 4-6), and biasing mechanisms 334a, 334b (also, collectively “biasing mechanisms 334”) It is to be understood that more than one biasing mechanism 334 can be provided. When a tissue T is loaded into the tissue cartridge 300 as described in reference to FIGS. 7A-C, the tissue cartridge 300 also includes a tissue T and a return plate 340. In the embodiments shown, biasing mechanisms 334 are springs each having a first end 344 and a second end 346. Biasing mechanisms 334 can be various types of springs and take other forms as well. The springs 335 have a first, unbiased position tending to push the doors 330 in a direction outward with respect to a central longitudinal axis CA of the tissue cartridge 300 (FIG. 2). In other words, as with the springs 335, the doors 330 can be said to have a first, unbiased position. The springs are configured to hold the doors 330 in a second, biased position via a clip 315 as described more fully herein below. The tissue engagement mechanism or mechanisms 332 extend downwardly from a lower or bottom surface 338a, 338b (also, collectively, “lower surfaces 338”) of doors 330 and can be removable from or incorporated into doors 330. Tissue engagement mechanisms 332 likewise may take a variety of forms including that of a pin, spike, hook, anchor or any other structure or fastening element which effectively holds a test sample or tissue T to be tested. Alternatively, lower surfaces 338 of doors 330 can frictionally engage tissue T such as via a patterned, grooved, or raised “bump” type of surface. As a further alternative, a clamp may be used to clamp tissue T in place. Regardless of the manner in which tissue T is engaged or held by doors 330 the tissue engagement mechanism or mechanisms 332 are configured to engage or hold tissue T in a manner which allows for pulling or tensioning of the tissue T, discussed more fully below.

Referring now between FIGS. 4, 5 and 6, the doors 330 can be constructed of a variety of materials including stainless steel, a polymeric material or materials, or any other suitable material or combination of materials. The doors 330 have sidewalls 311a, 311b (FIG. 6) which may include a chamfered portion 314, as shown. Chamfered portion 314 may aid in more readily accessing or viewing a mounted tissue or test sample T. Each door 330 includes a door arm 312a, 312b (collectively, “door arms 312”) that extends along opposed sides 324 of a channel plate 320. A channel 322 is formed at an approximate center portion of channel plate 320. Doors 330 are slidably attached to opposed top walls 383 of channel plate 320 via door channels 360 (FIG. 4). Channel screws 362 act as stops to prevent travel of doors 330 past a desired position. As best seen in FIG. 6, the biasing mechanisms 334 are provided in a recess 380 formed by walls 381, 382 and 383 of channel plate 320. A first end 344 of each of springs 335 abuts an inner surface of a door arm 312 while a second end 346 of each of springs 335 abuts an inner surface of the wall 382 of channel plate 320 such that contact of ends 344 with the inner surfaces of door arms 312 allows for the outward movement of the doors 330 described above.

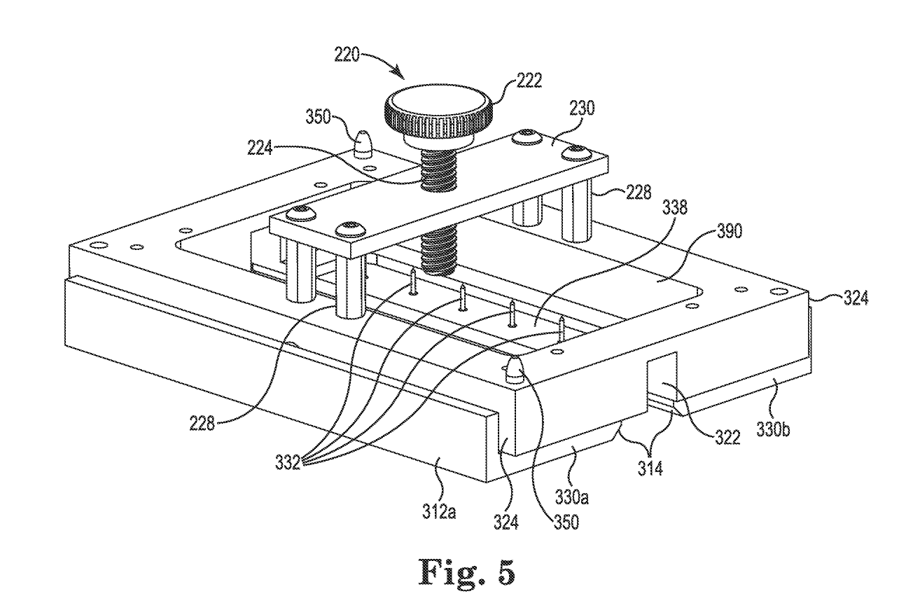

As best illustrated in FIGS. 5 and 6, the return plate adjuster 220 is threadably coupled to an adjuster positioning plate 230 and includes a return plate adjuster knob 222 and a return plate adjuster shaft 224. The adjuster positioning plate 230 is coupled to channel plate 320 via positioning plate anchors 228. In the illustrative embodiment, anchors 228 are screws, though anchors 228 may take other forms as well and as contemplated by this disclosure. An adjuster shaft distal end 221 is configured to selectively abut or contact the return plate 340, such as shown in FIG. 6, to aid in holding the return plate 340 in place as will be explained more fully herein below with reference to FIGS. 7A-C. To effect movement of the return plate adjuster 220, the adjuster knob 222 is turned by a hand of a user in a clockwise or counterclockwise motion, allowing shaft 224 to travel toward or away from the return plate 340. When the return plate adjuster 220 contacts the return plate 340, the return plate 340 may be moved toward doors 330 thereby applying an upward force F (relative to a bottom wall B of housing 210) on the tissue T. In other words, the return plate 340 can be moved or tensioned upward such that the return plate 340 contacts an underside of the tissue T and can effectively aid in holding or clamping the tissue T in place. Return plate 340 also provides a return path for energy (such as radiofrequency energy) delivered to the tissue T from a device 20. As can be seen in FIGS. 7A-C, a return plate connector 342 connects the return plate to ground.

Generally speaking, the tissue cartridge 300, and in particular, the tissue cartridge doors 330, are configured to apply tension to tissue T while a function of the device 20 (such as cutting or cauterizing) is tested on the tissue T. Mounting or loading of tissue T into the tissue cartridge 300 will now be described with reference to FIGS. 7A-C. When the tissue cartridge 300 is separated or removed from the housing 210, such as depicted in FIGS. 4, 5 and 7A-C, a tissue sample T may be loaded onto the tissue cartridge 300 from the bottom of the tissue cartridge 300. Thereafter, the tissue cartridge 300 is placed back onto the housing 210, and the entire tissue carriage 200 is coupled to the linear actuator (i.e., the carriage 200 is coupled to the first actuator travel carriage 512 as described above).

FIG. 7A depicts tissue cartridge 300 flipped or turned bottom side up (relative to final assembly of tissue cartridge 300 when placed in tissue carriage 200, e.g., FIGS. 1-3 and 6). After the tissue cartridge 300 is removed from the housing 210 and flipped as shown in FIGS. 5 and 7A-7B, a clip 315 is placed around arms 312 of doors 330 to hold doors 330 slightly inwardly or in a direction D3 as shown in FIG. 6. In this manner, clip 315 biases the springs 335 and doors 330 to a second, biased position. Stated another way, the clip 315 effectively “pre-loads” the springs 335 by pushing arms 312 against ends 344 of the springs 335. The clip 315 may be made of a variety of materials including stainless steel or other metals or polymeric materials. This “pre-loading” of the biasing mechanisms or springs 335 allows for later release of the pre-loaded force (i.e., return of the springs 335 to the first or unbiased state) which in turn allows the doors 330 to move outwardly (shown by arrows D4) with respect to the central longitudinal axis CA and thus pulls the issue T, as explained further below. A tissue sample T, for example an excised animal skin patch, is then placed, skin side (or desired testing side) down, in a central recess 390 of channel plate 320. In other words, the top surface TS of the tissue T is placed to face or abut the tissue engagement mechanism or mechanisms 332. In some of the illustrated embodiments, the tissue engagement mechanisms 332 are a plurality of spikes 332. After the tissue T is placed in recess 390, the return plate 340 is positioned on the underside or lower surface LS of the tissue T as shown in FIG. 7B. As can be seen, the return plate 340 is slid under positioning plate 230 and contacts the lower surface LS (FIG. 6) of the tissue T. The amount of surface of the tissue T which is contacted by the return plate 340 and the size of the return plate 340 are to be such that adequate electrical contact is made when a device 20 therapy is applied to tissue T and such that the return plate 340 is sufficiently sized to support or clamp in place the tissue T. A user may then manually press on the return pad 340 to fully engage the spikes 332 or other tissue engagement mechanisms 332 into the tissue T. For example, a user may use two fingers (e.g., thumbs) to press on opposed sides of return pad 340 which correspond to the area above the spikes 332 Pressing the tissue T onto the tissue engagement mechanisms 332 causes the top surface TS of the tissue T to abut the lower surface 338 of each of doors 330 as is clearly illustrated in, for example, FIG. 6.

Next, knob 222 may be turned to tighten the return plate adjuster 220 and hold the return plate 340 in place as described above. The return plate adjuster 220 may be turned or adjusted an amount sufficient to hold the return pad 340 and therefore the tissue T in place but still allow for the springs 335 to pull on the tissue T after the clip 315 is removed. Once the desired tension or force F is placed on the return pad 340 and tissue T, the tissue cartridge 300 can then be flipped over or flipped right side up and the clip 315 can be removed such as shown in FIG. 7C. Once the clip 315 is removed, the springs 335 are allowed to return to the original, first or unbiased position. That is, springs 335 are allowed to move outwardly in a direction D4 (FIG. 6) (or, outwardly with respect to a central longitudinal axis CA of cartridge 300) and in turn doors 330, having tissue T engaged by engagement mechanisms 332, are allowed to pull the tissue T thereby applying tension or force to the tissue T in the direction D4. In this manner the tissue T may be pulled taut. Once the clip 315 is removed, the loaded tissue cartridge 300, including tissue T and return plate 340, may then be attached to housing 210 via housing attachment elements 350 as described above, to provide a loaded tissue carriage 200. Tissue carriage 200 may then be coupled to the first actuator travel carriage 512 as described above.

As described above, a tissue sample T is can be mounted or loaded from the bottom. Bottom loading of the tissue T in this way allows the top surface TS of the tissue to be maintained at a generally consistent height with respect to the device 20. Providing the tissue T at a generally consistent height may allow for a more accurate depth of cut through multiple tissue samples T.

Once the return plate 340 is situated on the tissue T, as described above, the entire loaded tissue cartridge 300 is coupled to the housing 210 via housing attachment elements 350 as described above and as shown in FIG. 7D (the return plate connector 342 not being shown in FIG. 7D).

With reference back to FIGS. 1-3, once a loaded tissue carriage 200 is coupled to the linear actuator (e.g., attached to actuator travel carriage 512), and a device 20 is mounted to the device mount 400, the tissue T may be moved in a controlled or regulated manner (i.e. at a predetermined or known speed) past the device 20 via the linear actuator 500. Actuator 500 includes a stepper motor 520. A user can input a desired tissue travel speed (thus, a desired speed of cut), via the controller 30, which can include, for example a PC. Upon actuation, the actuator travel carriage 512 and thus the attached tissue carriage 200 travels at a the regulated or known speed along the actuator shaft 510. For example, FIG. 1 shows the actuator travel carriage 512 in a first position relative to the device 20 and FIG. 2 shows the actuator travel carriage 512 after the carriage 512 has traveled a distance along shaft 510 such that the tissue carriage 200 (and thereby the tissue cartridge 300) is being drawn past an end of device 20. Regulating or predetermining the speed of a cut or other test therapy aids in obtaining more accurate tissue depth of therapy effect data. For example, when a device 20 is on tissue T longer, the thermal damage or other effect can be deeper into the tissue T. Therefore, a slower application of therapy (e.g., cutting) can deliver more thermal damage to tissue T than a faster application of the same therapy. Knowing and having the ability to regulate the speed of the applied therapy can provide a more consistent and accurate method of measuring the thermal effects of an electrocautery incision.

In certain embodiments, a force transducer 600 can also be incorporated into the test fixture 10 for measuring the force required to cut the tissue T. Force data can show how easily or difficultly a device 20 cuts through tissue T. FIGS. 1-3 depict a force transducer 600 in contact with the tissue cartridge 300 via a sensor 620. Force transducer 600 is mounted to a second actuator travel carriage 513 which, as with carriage 512, is slidably mounted to the actuator shaft 510. Travel carriage 513 is configured to travel at the same speed as travel carriage 512 upon actuation of the linear actuator 500. An output, such as included with controller 30 can then show the force measured by the force transducer 600.

The entire fixture 10 can be provided on a text fixture base 30 for ease in transporting the fixture 10. Base 30 can be constructed of a variety of materials including but not limited to metals or polymers.

Embodiments according to the disclosure, as described above, can regulate testing input parameters, for example, parameters such as speed of incision (or other therapy), position of a device effective end as well as the tension applied to a tissue being tested. These parameters have historically been controlled by hand. Testing using the devices and methods described herein can provide for more accurate test data and a better understanding of the functions and capabilities of a test device.

Although the present disclosure has been described with reference to preferred embodiments, workers skilled in the art will recognize that changes can be made in form and detail without departing from the spirit and scope of the present disclosure.

What is claimed is:

1. A test fixture for an electrosurgical device, the test fixture comprising:
a tissue carriage comprising a tissue cartridge for loading a tissue sample;
a device mount for mounting the electrosurgical device;
a linear actuator for regulating a tissue incision speed.

2. The test fixture of claim 1 further comprising a force transducer for measuring a tissue incision force.

3. The test fixture of claim 1, wherein the tissue carriage is separably attached to a tissue carriage plate and the tissue cartridge is separably attached to a housing of the tissue carriage.

4. The test fixture of claim 2, wherein the tissue cartridge comprises a first door and a second door, each of the first and second doors comprising at least one tissue attachment mechanism;
at least two biasing mechanisms each biasing mechanism having a first, unbiased position and a second, biased position wherein in the second position; the biasing mechanisms are configured to bias the first and second doors inwardly with respect to a central longitudinal axis of the tissue cartridge and in the first position are configured to allow the doors to move outwardly with respect to the central longitudinal axis of the tissue cartridge; and
a return plate configured to act as a return path for an energy applied to the tissue sample.

5. The test fixture of claim 3, wherein the tissue engagement mechanisms comprise one of a pin or a spike and the biasing mechanisms comprise a spring.

6. The test fixture of claim 4, wherein the tissue cartridge comprises a return plate adjuster comprising an adjustment knob and a threaded adjuster shaft, wherein a distal end of the adjuster shaft is configured to engage the return plate and apply an adjustable upward force thereto.

7. The test fixture of claim 4, wherein each of the first and second doors comprise an upper surface and a lower surface and wherein the tissue sample is adapted to be loaded onto the tissue cartridge from the lower surfaces of the doors.

8. The test fixture of claim 1, wherein the device mount comprises an arm, a device mounting block, a device height adjuster, and a device rotational adjuster.

9. The test fixture of claim 7, wherein the device mounting block comprises a first and a second device holder plate configured to fixedly hold an electrosurgical device.

10. The test fixture of claim 1, wherein the tissue carriage is coupled to the linear actuator and is configured to move at a predetermined speed input by a user of the text fixture.

11. The test fixture of claim 9, wherein the tissue carriage is removably mounted to an actuator shaft of the linear actuator and is configured to move linearly along the actuator shaft at the predetermined speed, upon actuation;
wherein the device mount is configured to fixedly mount an electrosurgical device such that a distal end of the electrosurgical device is held at a fixed location relative to the tissue sample;
wherein the force transducer is connected to the tissue cartridge; and
wherein upon actuation of the linear actuator, the tissue carriage is configured to move such that the tissue sample is drawn past the distal end of the electrosurgical device.

12. A method of testing an electrocautery device, the method comprising:
tensionally loading a tissue sample in a tissue cartridge;
coupling the tissue cartridge to a linear actuator,
fixedly mounting the electrocautery device, such that an electrode of the electrocautery device is held at a fixed position relative to the tissue sample,
drawing the tissue sample past the electrode at a predetermined speed to thereby cut the tissue.

13. The method of claim 11, wherein tensionally mounting the tissue sample comprises loading a tissue sample from a bottom of a tissue cartridge such that a top surface of the tissue sample maintains an approximately consistent height with respect to the electrode).

14. The method of claim 12, wherein tensionally mounting the tissue sample further includes pushing the tissue sample into a plurality of tissue attachment mechanisms projecting from a bottom surface of a first and a second door of the tissue cartridge.

15. The method of claim 13, wherein tensionally mounting the tissue sample further includes allowing a biasing mechanism associated with each of the first and second doors to return to a first unbiased position such that the tissue sample is pulled in a direction outward relative to a central longitudinal axis of the tissue cartridge.

16. The method of claim 11, further comprising measuring the force to cut the tissue.

17. A system for testing the effects of an electrosurgical device on a tissue sample, the system comprising:
a text fixture comprising a tissue carriage connected to a linear actuator;
a device mount for fixedly mounting the electrosurgical device relative to the tissue sample;
an electrosurgical device mounted in the device mount;
a force transducer; and
a controller for controlling the speed of the linear actuator.

18. The system of claim 17, wherein the tissue carriage comprises a tissue cartridge including a plurality of tissue engagement mechanisms, at least two biasing mechanisms and an electrically conductive return plate;
wherein the device mount comprises a detachably connected device mounting block, a device height adjuster, and a device rotational adjuster;
wherein the device mount is configured to hold the electrosurgical device at a fixed angle and distance relative to the tissue sample; and
wherein the linear actuator is configured to draw the tissue sample past a tissue effecting end of the electrosurgical device at a user-input speed whereby the tissue is cut as it passes the tissue effecting end; and
wherein the force transducer is configured to measure the force to cut the tissue.

19. The system of claim 18, wherein the tissue cartridge is separably attached to a housing of the tissue carriage and is configured for bottom loading of a tissue sample.

20. The system of claim 17, wherein the electrosurgical device comprises one of a plasma blade, scalpel, and pencil
